(19) United States
(12) Patent Application Publication
(10) Pub. No.: US 2007/0219115 A1
(43) Pub. Date: Sep. 20, 2007

(54) Pharmaceutical Uses of Bisphosphonates

(76) Inventors: Robert Edward Coleman, Sheffield (GB); Ingunn Holen, Sheffield (GB); Helen Neville-Webbe, Sheffield (GB); Catherine Alyson Evans, Sheffield (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/578,290

(22) PCT Filed: Dec. 2, 2004
(86) PCT No.: PCT/EP04/13728

§ 371 Date: Jan. 11, 2007

(30) Foreign Application Priority Data
Dec. 3, 2003 (GB) 0328040.1

(51) Int. Cl. A61K 31/675; A61K 31/16; A61K 31/21; A61K 38/16; A61K 31/4196; A61K 31/663

(52) U.S. Cl. 514/2; 514/108; 514/383; 514/511; 514/94

(57) ABSTRACT

A method of treating a patient suffering from a malignant disease comprising administering to the patient an effective amount of a chemotherapeutic agent selected from the group consisting of: taxol, a derivative thereof and letrozole, followed sequentially by an effective amount of a bisphosphonate. The invention further provides a method of treating a patient suffering from a malignant disease comprising administering to the patient an effective amount of a bisphosphonate followed sequentially by an effective amount of TRAIL.

This invention relates to bisphosphonates, in particular to new pharmaceuticals uses of, and treatment methods including bisphosphonates.

Bisphosphonates are widely used to inhibit osteoclast activity in a variety of both benign and malignant diseases, which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity of osteoclasts.

Recent studies have also shown that some bisphosphonates may have a direct effect on tumour cells. Thus for example it has been found that relatively high concentrations of bisphosphonates, including zoledronate, induce apoptosis of breast and prostate carcinoma and myeloma cells in vitro (Senaratne et al. Br. J. Cancer, 82: 1459-1468, 2000; Lee et al., Cancer Res., 61: 2602-2608, 2001, Shipman et al. Br. J. Cancer, 98: 665-672 (1997)).

It has now been found that if certain types of bisphosphonates are used in combination with an additional chemotherapeutic agent to treat cancer cells in vitro, that enhanced, and in some cases synergistic, cell growth inhibition is achieved compared with use of either the bisphosphonate or chemotherapeutic agent alone. Suitable chemotherapeutic agents include taxols or derivatives thereof, aromatase inhibitors (e.g. letrozole), and TNF-related apoptosis inducing ligand (TRAIL). In particular, the ability of particular combinations of the commonly used chemotherapy agent Paclitaxel (PAC) and the potent anti-resorptive agent Zoledronic acid (ZOL) when administered sequentially, letrozole and ZOL when administered sequentially, and TRAIL and ZOL when administered sequentially, to induce apoptosis of breast and prostate cancer cells in vitro has now been established. The order in which the agents are given significantly affects the maximum level of apoptosis achieved.

The invention provides a method of treating a patient suffering from a malignant disease comprising administering to the patient an effective amount of a chemotherapeutic agent selected from the group consisting of: taxol or a derivative thereof or an aromatase inhibitor; followed sequentially by an effective amount of a bisphosphonate.

The invention further provides a method of treating a patient suffering from a malignant disease comprising administering to the patient an effective amount of a bisphosphonate followed sequentially by an effective amount of TRAIL.

Yet further the invention provides the sequential use of a chemotherapeutic agent selected from the group consisting of: taxol, a derivative thereof, an aromatase inhibitor, and TRAIL; and a bisphosphonate to inhibit cancer cell growth or induce cancer cell apoptosis.

Yet further the invention provides the use of a bisphosphonate in the manufacture of a medicament for the treatment of malignancies in a patient already receiving a chemotherapeutic agent selected from the group consisting of: taxol, a derivative thereof, an aromatase inhibitor and TRAIL.

Yet further the invention provides the use of a chemotherapeutic agent selected from the group consisting of: taxol, a derivative thereof, an aromatase inhibitor and TRAIL; in the manufacture of a medicament for the treatment of malignancies in a patient already receiving a bisphosphonate.

Accordingly in a yet further aspect the present invention also provides a pharmaceutical preparation for treatment of malignancies, which comprises a chemotherapeutic agent selected from the group consisting of: taxol, a derivative thereof, an aromatase inhibitor, and TRAIL; and a bisphosphonate for sequential use.

Yet further the invention provides a commercial package comprising a unit dosage form of a bisphosphonate or a pharmaceutically acceptable salt thereof, or any hydrate thereof, and a unit dosage form of a chemotherapeutic agent selected from the group consisting of: taxol, a derivative thereof, an aromatase inhibitor and TRAIL; together with instructions for administering sequential unit doses of said chemotherapeutic agent and said bisphosphonate for the treatment of malignant diseases.

Yet further the invention provides a kit comprising a unit dosage form of a bisphosphonate or a pharmaceutically acceptable salt thereof, or any hydrate thereof, and a unit dosage form of a chemotherapeutic agent selected from the group consisting of: taxol, a derivative thereof, an aromatase inhibitor and TRAIL; together with instructions for administering sequential unit doses of said chemotherapeutic agent and said bisphosphonate for the treatment of malignant disease.

In the present description the term “teatment” includes both prophylactic or preventative treatment as well as curative or disease modifying treatment, including treatment of patients at risk of contracting the disease or suspected to have contracted the disease as well as ill patients.

The invention is generally applicable to the treatment of malignant diseases for which bisphosphonate treatment is indicated. Thus typically the disease is a malignant disease which is associated with the development of bone metastases or excessive bone resorption. Examples of such diseases include cancers, such as breast and prostate cancers, multiple myeloma (MM), tumour induced hypertension (TIH) and similar diseases and conditions. In particular the invention is applicable to the treatment of bone metastases (BM) associated with cancers such as breast cancer, lung cancer, colon cancer or prostate cancer.

The compositions, uses and methods of the present invention represent an improvement to existing therapy of malignant diseases in which bisphosphonates are used to prevent or inhibit development of bone metastases or excessive bone resorption, and in which (as has been discovered in accordance with the present invention) bisphosphonate treatment also inhibits cancer cell growth or induces cancer cell apoptosis. The sequential use of a chemotherapeutic agent such as paclitaxel or letrozole and a bisphosphonate advantageously gives rise to enhanced, or even synergistic, levels of cancer cell growth inhibition or cancer cell apoptosis.

The bisphosphonates for use in the present invention are preferably N-bisphosphonates.

For the purposes of the present description an N-bisphosphonate is a compound which in addition to the characteristic geminal bisphosphate moiety comprises a nitrogen containing side chain, e.g. a compound of formula I 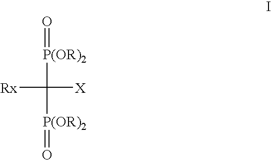
wherein
X is hydrogen, hydroxyl, amino, alkanoyl, or an amino group substituted by C₁-C₄ alky, or alkanoyl;
R is hydrogen or C₁-C₄ alkyl and
Rx is a side chain which contains an optionally substituted amino group, or a nitrogen containing heterocycle (including aromatic nitrogen-containing heterocycles),
and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II 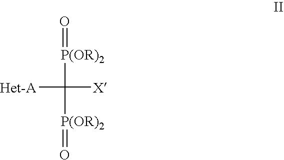
wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a particularly preferred bisphosphonate for use in the invention comprises a compound of Formula III 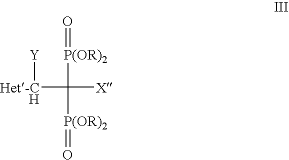
wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a particularly preferred bisphosphonate for use in the invention comprises a compound of Formula IV 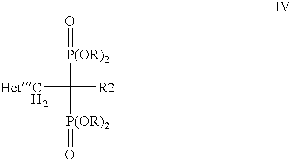
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mono- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.

Examples of particularly preferred N-bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof.

All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The bisphosphonates can also be used in the form of their hydrates or include other solvents used for their crystallisation.

Pharmacologically acceptable salts of bisphosphonates are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts of the bisphosphonates are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts of the bisphosphonates is characterized by having one acidic hydrogen and one pharmaceutically acceptable cation, especially sodium, in each of the phosphonic acid groups.

The bisphosphonates are preferably used in the form of pharmaceutical compositions that contain a therapeutically effective amount of active ingredient optionally together with or in admixture with inorganic or organic, solid or liquid, pharmaceutically acceptable carriers which are suitable for administration.

The bisphosphonate pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the bisphosphonate pharmaceutical compositions are adapted to oral or parenteral (especially intravenous, intra-arterial or transdermal) administration. Intravenous and oral, first and foremost intravenous, administration is considered to be of particular importance. Preferably the N-bisphosphonate active ingredient is in a parenteral form, most preferably an intravenous form.

The particular mode of administration and the dosage may be selected by the attending physician taking into account the particulars of the patient, especially age, weight, life style, activity level, and disease state as appropriate. Most preferably, however, the bisphosphonate is administered intravenously.

The dosage of the bisphosphonate for use in the invention may depend on various factors, such as effectiveness and duration of action of the active ingredient, mode of administration, warm-blooded species, and/or sex, age, weight and individual condition of the warm-blooded animal.

Taxol is the compound [2aR-[2aα, 4β, 4αβ, 6αβ, 9α(αR*, βS*),-11α,12α,12aα,12bα]]-β-(benzoylamino)-α-hydroxybenzenepropanoic acid 6,12b-bis(acetyloxy)-12-(benzoyloxy)-2α,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester, alternatively known as Paclitaxel, which is an antileukemic and antitumour agent, first isolated as the 1-form from the bark of the Pacific yew tree, Taxus brevifolia, Taxaceae. Suitable derivatives of taxol for use in the present invention include taxotere (i.e. the compound [2aR-[2aα, 4β, 4αβ,6β,9α(αR*, βS*),-11α,12α, 12aα, 12bα]]-β-[[(1,1-dimethylethoxy)carbonyl]-amino]-α-hydroxybenzenepropanoic acid 12b-(acetyloxy)-12-(benzoyloxy)-2α,3,4,4a,5,6,9,10,11,12,12a,12b-dodecahydro-4,6,11-trihydroxy-4a,8,13,13-tetramethyl-5-oxo-7,11-methano-1H-cyclodeca[3,4]benz[1,2-b]oxet-9-yl ester, alternatively known as docetaxel), taxanes, taxines (e.g. taxine I, taxine II, taxine A or taxine B) or any other suitable taxol derivative. Taxol and suitable derivatives thereof may be used in combination with a bisphosphonate in the present invention. Paclitaxel (PAC) is a preferred taxol derivative for use in the present invention. The taxol or taxol derivative pharmaceutical composition may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN.

The preferred aromatase inhibitor according to the invention is letrozole. Letrozole is the compound 4-[α-(4-cyanophenyl)-1-(1,2,4-triazolyl)methyl]-benzonitrile. Letrozole can be administered in the form as it is marketed, e.g. under the trade mark FEMARA™ or FEMAR™ or by any other suitable means, e.g. as a composition for enteral, such as oral, rectal, aerosol inhalation or nasal administration, compositions for parenteral, such as intravenous or subcutaneous administration, or compositions for transdermal administration (e.g. passive or iontophoretic).

The Agents of the Invention (a taxol or derivative thereof, letrozole or TRAIL and b. the bisphosphonate) are used in the form of separate pharmaceutical preparations that each contain the relevant therapeutically effective amount of the respective active ingredient optionally together with or in admixture with inorganic or organic, solid or liquid, pharmaceutically acceptable carriers which are suitable for administration.

The particular mode of administration and the dosage may be selected by the attending physician taking into account the particulars of the patient, especially age, weight, life style, activity level, etc.

The dosage of the Agents of the Invention may depend on various factors, such as effectiveness and duration of action of the active ingredient, mode of administration, warm-blooded species, and/or sex, age, weight and individual condition of the warm-blooded animal.

The pharmacologically active compounds of the invention are useful in the manufacture of pharmaceutical compositions comprising an effective amount thereof in conjunction or admixture with excipients or carriers suitable for either enteral or parenteral application. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient.

Tablets may be either film coated or enteric coated according to methods known in the art.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like. Such topical delivery systems will in particular be appropriate for dermal application, e.g. for the treatment of skin cancer, for example, for prophylactic use in creams, lotions sprays and the like.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following examples are intended to illustrate the invention and are not to be construed as being limitations thereon.

### EXPERIMENTAL

Method 1: PAC and ZOL

MCF7 cells are seeded and pre-incubated then treated with ZOL and/or PAC in the sequences given below. In the case of the PAC then ZOL sequence, in one of the experiments geranylglycerol (GGOH, 50 μM) is added and then removed with the ZOL. Apoptosis is determined by evaluation of nuclear morphology.

Experiment 1:

Group 1: 25 μM ZOL (for 1 hour on day 1) followed by 2 nM PAC (for 4 hours day 2)

Group 2: 2 nM PAC (for 4 hours on day 1) followed by 25 μm ZOL (for 1 hour on day 2)

Following the last drug exposure the cells in each group are incubated in drug free medium to 48 hours.

Experiment 2:

2 nM PAC (for 4 hours on day 1) followed by 1 μM ZOL (for 1 hour on day 2). Following the last drug exposure the cells are incubated in drug free medium to 48 hours.

Experiment 3:

2 nM PAC (for 4 hours on day 1) followed by 25 μM or 1 μM ZOL (for 1 hour on day 2), with or without geranylgeraniol (GGOH) 50 μM (added and removed simultaneously with the ZOL). Following the last drug exposure the cells are incubated in drug free medium to 48 hours.

Results

Experiment 1 Results:

Giving 25 μM ZOL before PAC (Group 1) induces 2.4% apoptosis, whereas maximal induction of apoptosis is seen in cells treated with PAC on day 1 followed by ZOL (25 μM) on day 2 (Group 2); (6.1%, p<0.001 compared to ZOL or PAC alone).

From these results it is shown that apoptosis is induced in a synergistic fashion but the order of drug exposure is important for maximal apoptosis.

Experiment 2 Results:

As is found using higher doses, MCF7 cells treated with PAC (day 1) followed by 1 μM ZOL (day 2) also induces apoptosis in a synergistic fashion, with 4.1% in the combined group, compared to PAC alone (1.25%, p=0.004) and 1 μM ZOL alone (0.25%, p=0.004). These results demonstrate that clinically relevant doses of ZOL are effective at inducing apoptosis. Following infusion of ZOL in vivo peak plasma concentration is 1-2 μM for a few hours.

Experiment 3 Results:

Geranylgeraniol is an intermediary of the mevalonate pathway which is able to reverse the effects of ZOL. Treating MCF7 cells with PAC followed by ZOL combined with geranylgeraniol (GGOH, 50 μM) prevents the synergistic increase in apoptotic cell death by 70-80%. This shows that ZOL induces apoptosis of MCF7 cells via inhibition of the MVA pathway.

Discussion

Combinations of ZOL and PAC are shown to have a synergistic effect in inducing apoptosis. Additionally, it is found that synergy is achievable with shorter incubation periods and clinically relevant concentrations of ZOL. For maximal induction of apoptosis cells must be exposed to PAC first followed by ZOL, preferably on separate days. Induction of apoptosis is via inhibition of the MVA pathway. Our results suggest that combining PAC and clinically relevant doses of ZOL does induce apoptosis of tumour cells, and that the drug sequence is important for obtaining maximum effect of combined treatment.

Method 2: ZOL and TRAIL

The breast cancer lines MDA-MB-426 and MCF7 and the prostate cancer cell line PC3 are treated with ZOL (25 μM and TRAIL (purchased from R&D systems, Abingdon, UK; 10 ng/ml) for varying incubation periods and sequences as follows:

Group 1: TRAIL given first for 24 hours followed by ZOL for 48 hours.

Group 2: ZOL given first for 24 hours followed by TRAIL for 24 hours.

Group 3: ZOL and TRAIL given simultaneously for 24 hours.

Group 4: TRAIL given first for 24 hours then ZOL for 4 hours followed by drug-free medium for 48 hours.

Group 5: ZOL given first for 4 hours and cells maintained in drug-free medium for 48 hours followed by TRAIL for 24 hours.

Results

The combination of ZOL and TRAIL is shown to have synergistic effects in inducing apoptotic death of breast cancer cells, but the order in which the drugs are given is significant. Giving TRAIL before ZOL (group 1) increases apoptosis from 1.75% (TRAIL only) and 0.5% (ZOL only) to 2.4% in the combined group. Likewise, treating with ZOL and TRAIL together for 24 hours (group 3) increases the level of apoptosis from 1.75% (TRAIL only) and 0.7% (ZOL only) to 2.5% in the combined group. When cells are treated with ZOL for 48 hours followed by TRAIL (group 2), the results indicate synergy between these two drugs. In the combined group there is 14.650% apotosis which is significantly greater than ZOL only, (0.7% p<0.001) and TRAIL only (2.7% p<0.001). Similar results are obtained when a shorter incubation period with ZOL (4 hours) was used, with apparent synergy obtained only when ZOL is given before TRAIL. Prostate cancer cells are also sensitive to the combination of ZOL and TRAIL when these cells are treated as outlined for group 5.

These results show that the combination of ZOL and TRAIL have a synergistic effect in inducing apoptosis of tumour cells. For maximum effect ZOL has to be given prior to TRAIL treatment.

Method 3: Letrozole (LET) and ZOL

Materials and Methods:

Breast cancer cells MCF7Ca (gift from Dr Chen, NY, USA) are used. Letrozole and zoledronic acid are provided by Novartis AG, Basel, Switzerland.

The Following Studies are Performed:

1. The effects of zoledronic acid alone on MCF7Ca growth
2. The effects of letrozole alone on MCF7Ca growth
3. The effects of zoledronic acid alone on MCF7Ca apoptosis and necrosis
4. The effects of letrozole alone on MCF7Ca apoptosis and necrosis
5. The effects of combined treatment (zoledronic acid and letrozole together) on apoptosis of MCF7Ca
6. The effects of sequential treatment (zoledronic acid followed by letrozole) on apoptosis of MCF7Ca
7. The effects of sequential treatment (letrozole followed by zoledronic acid) on apoptosis of MCF7Ca
8. The effects of adding GGOH on the levels of apoptosis caused by letrozole followed by zoledronic acid
9. The effects of sequential and combined treatment in serum free medium
   Doses and Incubation Times Used:

Following the initial dose-response studies the sequential and combined treatments are carried out using 10 μM zoledronic acid and 100 nM letrozole for 24 hours each, followed by a drug-free incubation period up to 24 hours. In some additional studies 1 μM zoledronic acid is used for 1 hour only.

Measurements of Apoptosis/Necrosis:

The levels of apoptotic/necrotic tumour cell death are determined by evaluation of nuclear morphology following staining of the cells using Hoechst and propidium iodide.

Results:

1) The effects of Zoledronic Acid on Growth of MCF7Ca Cells

MCF7Ca cells are exposed to increasing doses of zoledronic acid for 1 hour, the drug removed and the cells incubated in drug-free medium up to 72 hours. The number of cells are counted using a coulter counter.

[image]

Doses of zoledronic acid above 1 μM cause a decrease in the number of MCF7Ca, whereas lower doses have no effect on cell growth. No significant difference in effect is seen between 1, 20 and 25 μM zoledronic acid.

2) The Effects of Letrozole on Growth of MCF7Ca Cells

MCF7Ca cells are exposed to increasing doses of letrozole for 24, 48 and 72 hours. For the 24 and 48 hour time points the drug is removed and the cells incubated in drug-free medium up to 72 hours. The number of cells are counted using a coulter counter.

[image]

The effects of letrozole on cell growth appears to be biphasic. Increased number of cells are counted when cells are treated with 0.1 nM letrozole (all time points), 1 nM (24 and 48 hours). Even after 72 hours incubation in the presence of 100 nM letrozole the inhibition of cell growth is moderate, with a 31% reduction in cell number compared to control.

3) The Effects of Zoledronic Acid on Apoptosis and Necrosis in MCF7Ca Cells

The levels of apoptosis and necrosis caused by increasing doses of zoledronic acid is determined by expressing the cells to zoledronic acid for 1 hour, followed by a 72 hour incubation in drug-free medium.

[image]

The MCF7Ca cells appeared to be relatively insensitive to zoledronic acid, with less than 3% apoptotic cells counted following exposure to 100 μM. The levels of necrotic cell death in the cultures is also low, maximum 4.5% following exposure to 100 μM.

4) The Effects of Letrozole on Apoptosis and Necrosis in MCF7Ca Cells

The levels of apoptosis caused by increasing doses of letrozole is determined by exposing the cells to letrozole for 72 hours.

[image]

The levels of both necrotic and apoptotic cell death in the cultures are low, with the highest dose tested (100 nM) causing less than 4% necrosis and less than 3% apoptosis.

5) The Effects of Combined Treatment Using Letrozole and Zoledronic Acid on Apoptosis of MCF7Ca Cells

Cells are treated with a combination of 100 nM letrozole and 10 μM zoledronic acid for 24 hours, and the levels of apoptotic cell death determined following an additional 48 hours incubation in drug-free medium.

[image]

There is no statistically significant increase in the level of apoptosis following this combined treatment.

6) The Effects of Sequential Treatment Using Letrozole and Zoledronic Acid on Apoptosis of MCF7Ca Cells

In this series of experiments the cells are exposed to 10 μM zoledronic acid for 24 hours, washed and subsequently treated with 100 nM letrozole for 24 hours. The levels of apoptotic cell death are determined following a further 24 hours incubation in drug-free medium.

[image]

No significant increase in the level of apoptotic cell death is observed when cells are treated with zoledronic acid followed by letrozole. If the sequence is reversed so cells are exposed to letrozole prior to zoledronic acid we observe a significant increase in the number of apoptotic cells in the cultures.

7) The Effects of Geraniylgeraniol (GGOH) on Apoptosis Caused by Sequential Treatment Using Letrozole Followed by Zoledronic Acid

In order to determine to what extent the observed increase in apoptosis following sequential treatment with letrozole and zoledronic acid is due to the effect of zoledronic acid, experiments are carried out where the cells are treated with 50 μM GGOH added at the same time as zoledronic acid. (GGOH is an intermediary of the mevalonate pathway and is commonly used to reverse the effects of zoledronic acid inhibition of this pathway). In this set of experiments cells are treated with 100 nM letrozole for 24 hours followed by 1 hour treatment with 10 μM zoledronic acid+50 μM GGOH. The level of apoptosis is determined at 72 hours.

All groups are significantly less than letrozole then zoledronic acid (p<0.05), whilst the group letrozole then zoledronic acid +GGOH has a level of apoptosis that is significantly lower than in the let then zol group (p=0.05).

[image]

By adding GGOH at the same time as zoledronic acid the level of apoptosis in the cultures is reduced to the same level observed using letrozole alone. These results suggest that the majority of the apoptotic effect is due to zoledronic acid.

8) Effects of Combined and Sequential Treatment in Serum Free Medium

These experiments are performed in order to determine if the effects of zoledronic acid and letrozole are affected by starving the cells by treating them under serum-free conditions.

[image]

When treating the cells in serum-free conditions a statistically significant synergistic increase in apoptotic cell death is observed for the cells treated with letrozole first followed by zoledronic acid.

### CONCLUSIONS

Zoledronic acid and letrozole do not induce significant levels of apoptotic cell death when MCF7Ca are treated with each drug individually.

Potential for synergistic induction of apoptosis is achievable, but is drug-sequence dependent. Administering zoledronic acid before or with letrozole resulted in 2.69% and 1.27% apoptosis respectively, whereas cells treated with ‘letrozole then zoledronic acid’ induced 9.21% apoptosis (p<0.05 compared to each drug alone).

1. A pharmaceutical preparation for treatment of malignancies, which comprises a chemotherapeutic agent selected from the group consisting of: taxol, a derivative thereof, and aromatase inhibitor and TRAIL; and a bisphosphonate for sequential use.

2. A pharmaceutical preparation according to claim 1 in which the bisphosphonate is an N-bisphosphonate.

3. A pharmaceutical preparation according to claim 1 in which the bisphosphonate is a compound of formula I 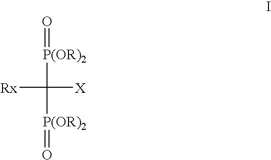
wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles), or a pharmaceutically acceptable salt thereof or any hydrate thereof.

4. A pharmaceutical preparation according to claim 1, in which the bisphosphonate is 2-(imidazol-lyl)-1-hydroxyethane 1,1-diphosphonic acid (zoledronic acid) or a pharmacologically acceptable salt thereof.

5. A pharmaceutical preparation according to claim 1, in which the chemotherapeutic agent is paclitaxel or letrozole.

6. A pharmaceutical preparation according to claim 1, in which the chemotherapeutic agent is TNF-related apoptosis inducing ligand.

7. A method of treating a patient suffering from a malignant disease comprising administering to the patient an effective amount of a chemotherapeutic agent selected from: taxol or a derivative thereof or letrozole; followed sequentially by an effective amount of a bisphosphonate.

8. A method according to claim 7 wherein the bisphosphonate is an N-bisphosphonate.

9. A method according to claim 7 wherein the bisphosphonate is a compound of formula I 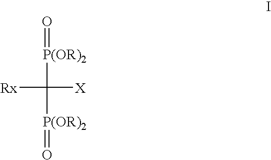
wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles), or a pharmaceutically acceptable salt thereof or any hydrate thereof.

10. A method according to claim 7 wherein the bisphosphonate is 2-(imidazol-lyl)-1-hydroxyethane-1,1-diphosphonic acid (zoledronic acid) or a pharmacologically acceptable salt thereof.

11. A method according to claim 7 wherein the chemotherapeutic agent is paclitaxel.

12. A method according to claim 7 wherein the chemotherapeutic agent is an aromatase inhibitor and is letrozole.

13. A method of treating a patient suffering from a malignant disease comprising administering to the patient an effective amount of a bisphosphonate followed sequentially by an effective amount of TNF-related apoptosis inducing ligand.

14. A method according to claim 13 wherein the bisphosphonate is 2-(imidazol-lyl)-1-hydroxyethane-1,1-diphosphonic acid (zoledronic acid) or a pharmacologically acceptable salt thereof.

15. The sequential use of a chemotherapeutic agent selected from the group consisting of: taxol, a derivative thereof, an aromatase inhibitor and TRAIL; and a bisphosphonate to inhibit cancer cell growth or induce cancer cell apoptosis.

16-25. (canceled)

26. A commercial package comprising a unit dosage form of a bisphosphonate or a pharmaceutically acceptable salt thereof, or any hydrate thereof, and a unit dosage form of a chemotherapeutic agent selected from the group consisting of: taxol, a derivative thereof, an aromatase inhibitor and TRAIL; together with instructions for administering sequential unit doses of said chemotherapeutic agent and said bisphosphonate for the treatment of malignant diseases.
